(19) United States
(12) Patent Application Publication
(10) Pub. No.: US 2017/0204357 A1
(43) Pub. Date: Jul. 20, 2017

(54) METHOD AND DEVICE FOR MAINTAINING MAMMALIAN AND MICROBIAL CELLS VIABLE AND INTACT DURING AMBIENT TEMPERATURE TRANSPORT AND STORAGE

(71) Applicant: Bassam El-Fahmawi, Union City, CA (US)

(72) Inventor: Bassam El-Fahmawi, Union City, CA (US)

(21) Appl. No.: 15/408,089

(22) Filed: Jan. 17, 2017

Related U.S. Application Data
(60) Provisional application No. 62/280,243, filed on Jan. 19, 2016.

(51) Int. Cl. C12M 1/00; C12M 1/24

(52) U.S. Cl. CPC C12M 23/52; C12M 23/08; C12M 23/38

(57) ABSTRACT

A device for maintaining the viability of mammalian and microbial cells during ambient temperature transport and storage may include a volume of a buffered sugar alcohol solution containing a sugar alcohol dissolved in nuclease free water; and a sealable vessel sized to accommodate the volume of the buffered sugar alcohol solution and a volume of cells to be transported and stored. In embodiments, the buffered sugar alcohol solution may have a pH of from about 6.0 to about 8.0.

### RELATED APPLICATION

This application claims priority to provisional patent application U.S. Ser. No. 62/280,243 filed on Jan. 19, 2016 entire contents of which is herein incorporated by reference.

### BACKGROUND

The embodiments herein relate generally to the transportation of biological cells and, more particularly, to a method and device for maintaining mammalian and microbial cells viable and intact during ambient temperature transport and storage.

Maintaining cells' viability in biological samples during transport for several days to week will be of significance in advancing cancer research and diagnosis, particularly for samples identified as liquid biopsies, which may include blood samples required for information to be gleaned from circulating tumor cells (CTCs), circulating tumor DNA (ctDNA) fragments, and extracellular vesicles (EVs).

However, cells typically do not retain their viability during transport unless they are frozen or dehydrated. Conventional stabilizing technologies for biological samples describe complex compositions that can either fix cells in a non-viable format (U.S. Patent Application Publication No. 2010/0184069 and U.S. Patent Application Publication No. 2011/0027771) or dehydrated format (U.S. Pat. No. 6,509,146). Alternatively, other publications, such as U.S. Pat. No. 6,602,718 and U.S. Pat. No. 6,617,120, describe lysing cells and stabilizing nucleic acids. Other publications describe using a mixture of salt, energy sources such as adenine, and mannitol (U.S. Pat. No. 4,267,269) or sorbitol and xylitol (U.S. Pat. No. 4,572,899) to help reduce hemolysis for packed erythrocyte transport. However, such stabilizer compositions are potentially only appropriate for cold storage for only erythrocytes at about 6° C. from collection to handling and have not been reported or proven to perform at ambient temperature from collection to processing. Moreover, the cost associated with the transport of viable cells using cold temperatures is about 600% more costly than transporting at standard ambient temperature.

Therefore, what is needed is a method and device for maintaining mammalian and microbial cells viable and intact during ambient temperature transport and storage.

### SUMMARY

Some embodiments of the present disclosure include a device for maintaining the viability of mammalian and microbial cells during ambient temperature transport and storage may include a volume of a buffered sugar alcohol solution containing a sugar alcohol dissolved in nuclease free water; and a sealable vessel sized to accommodate the volume of the buffered sugar alcohol solution and a volume of cells to be transported and stored. In embodiments, the buffered sugar alcohol solution may have a pH of from about 6.0 to about 8.0.

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of one embodiment of the present disclosure.

FIG. 2 is a flowchart of one embodiment of the present disclosure.

FIG. 3 is a flowchart of one embodiment of the present disclosure.

FIG. 4 is a chart showing exemplary buccal/mammalian cell stability.

FIG. 5 is a chart stabilizing microbial DNA.

FIG. 6 is a chart stabilizing microbial RNA.

FIG. 7 is a chart of a dual time point extracted DNA test.

FIG. 8 is a chart of a dual time point extracted gDNA test.

FIG. 9 is a schematic view of the mammalian cells collection device appearance 12 hours post-collection.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The method and device of the present disclosure may be used to transport mammalian and microbial cells at an ambient temperature, while simultaneously maintaining the viability of the cells, and may comprise the following elements. This list of possible constituent elements is intended to be exemplary only, and it is not intended that this list be used to limit the method or device of the present application to just these elements. Persons having ordinary skill in the art relevant to the present disclosure may understand there to be equivalent elements that may be substituted within the present disclosure without changing the essential function or operation of the device.

1. Vessel 2. Stabilizing Buffer

The various elements of the method and device of the present disclosure may be related in the following exemplary fashion. It is not intended to limit the scope or nature of the relationships between the various elements and the following examples are presented as illustrative examples only.

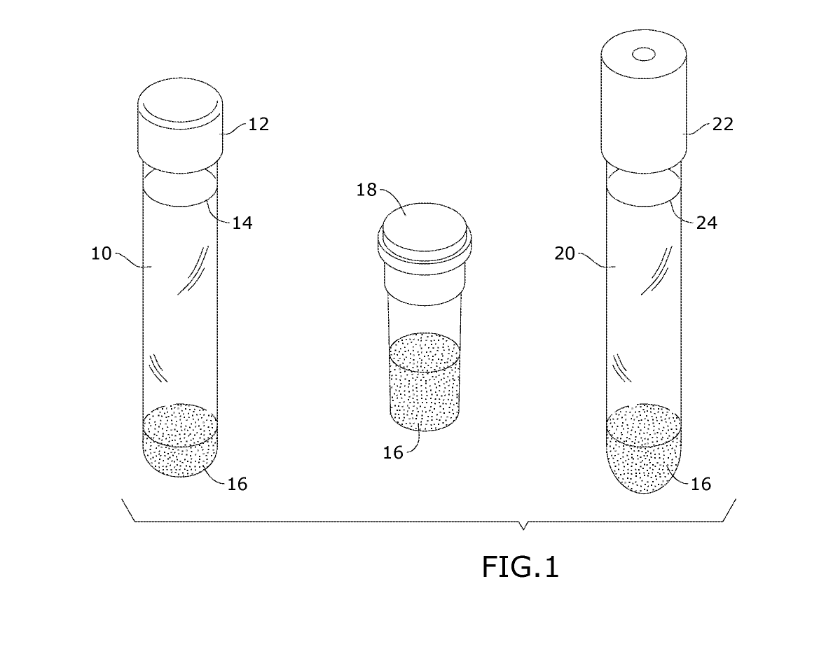

By way of example, and referring to FIGS. 1-9, some embodiments of the present disclosure include a method and device for maintaining the viability of mammalian and microbial cells during ambient temperature transport and storage, the device comprising a volume of a stabilizing buffer 16, and a vessel sized to accommodate the volume of stabilizing buffer 16 and a volume of cells.

In embodiments, the stabilizing buffer 16 may comprise a stabilizing buffer comprising a buffered sugar alcohol mixture, which may consist of sugar alcohols dissolved in nuclease free water. Suitable buffered sugar alcohol mixtures may have a pH ranging from about 5.0 to about 9.0. Additionally, suitable buffered sugar alcohol mixtures may comprise any compound that falls under polyhydric alcohols, polyalcohols, alditols, or glycitol. Specific examples of suitable buffered sugar alcohol solutions include glycerol, erythritol, threitol, arabitol, xylitol, ribitol, mannitol, sorbitol, galactitol, fucitol, iditol, inositol, volemitol, isomalt, malitol, lactitol, maltotriitol, maltotetraitol, and polyglycitol.

In embodiments, the vessel may comprise any suitable sealable vessel. For example, as shown in FIG. 1, the vessel may be a tube 10 with a spill proof cap 12, wherein the tube 10 may include a fill line 14 on an outer surface thereof. Alternatively, the vessel may comprise a conventional DNA collection device 18 or a vacuum tube 20 with a pierceable lid 22, wherein the vacuum tube 20 may include a fill line 24 on an outer surface thereof. While these exemplary vessels are shown in FIG. 1, other suitable vessels are also envisioned.

To use the system, a user may first create the stabilizing buffer 16, add a volume of the stabilizing buffer 16 to the vessel, and then collect a sample from a human or animal subjects. In embodiments, the ratio may be about 1 part stabilizing buffer 16 to about 3 to 4 parts sample.

In embodiments, the stabilizing device may be used by preparing the stabilizing buffer 16 in a proper vessel, wherein the vessel may be dependent on the sample type collected. As outlined in FIG. 3, preparing the stabilizing buffer 16 may comprise: filtering the buffer with a sterile filter, such as a 0.22 μm filter; dispensing the stabilizing buffer into individual screw cap tubes or air evacuated tubes; and closing the device with a screw cap or air tight pierceable cap. Using the device may comprised preparing the stabilizing buffer 16; obtaining mammalian or microbial cells; adding the collected sample to the vessel with a pre-aliquoted stabilizing buffer 16 or by releasing the collected sample from the swab by, for example, the method and device described in U.S. Pat. No. 9,138,205, the entire contents of which is herein incorporated by reference; and mixing the tube gently.

The biological sample may be collected with a swab or cytobrush for samples such as buccal, vaginal, wound, stool, soil samples, or the like. The tube may then be shipped at ambient temperature to, for example, a processing lab, wherein the processing lab may aliquot the stabilized viable cells or samples from the tube for further cellular and genetic analysis.

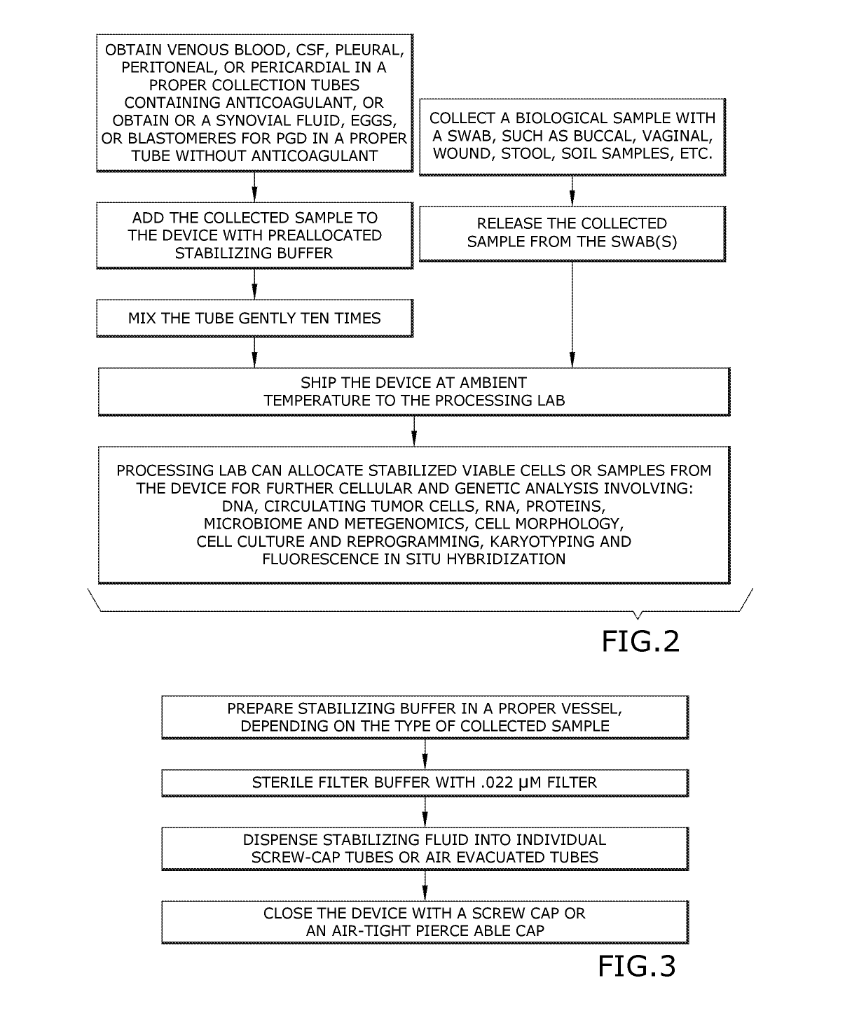

More specifically and as outlined in FIG. 2, the method of the present disclosure may comprise obtaining venous blood, cerebral spinal fluid (CSF), pleural, peritoneal, or pericardial in a proper collection tube containing anticoagulant or, alternatively, obtaining a synovial fluid, eggs, or blastomeres for preimplantation genetic diagnosis (PGD) in a proper collection tube without anticoagulant; adding the collected sample to the vessel with a pre-aliquoted stabilizing buffer 16; and mixing the vessel gently. Alternatively, a biological sample may be collected with a swab, wherein the sample may be buccal, vaginal, wound, stool, soil samples, or the like, and the sample may be released from the swab. In either case, the device may be shipped at ambient temperatures to, for example, a processing lab. The processing lab ma aliquot stabilized and viable cells or samples from the device for further cellular and genetic analysis, which could include DNA, circulating tumor cells, RNA, proteins, microbiome and metagenomics, cell morphology, cell culture and reprogramming, karyotyping and fluorescence, and in situ hybridization.

By using the method and device described herein, mammalian and microbial cells may be transported and stored at ambient temperature and maintain their viability for days to weeks.

### EXAMPLES

Viability Time Course:

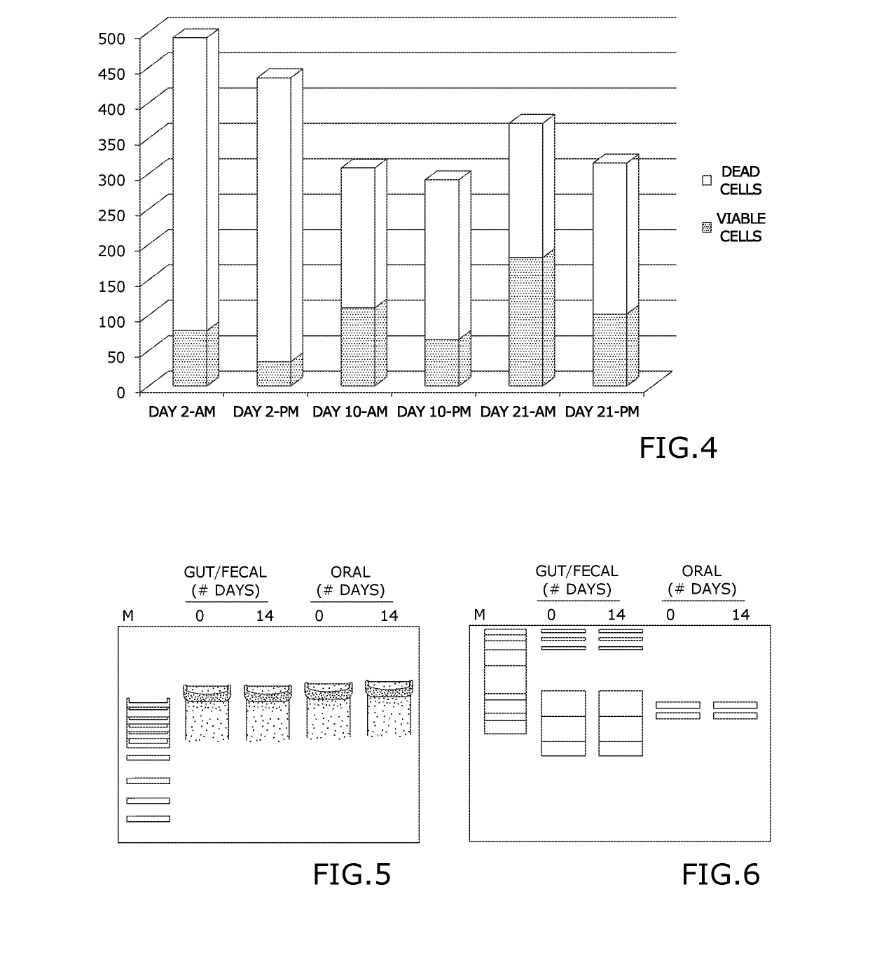

Buccal cells were collected from 6 volunteers (age 19-50). Each volunteer provided a sample early in the morning before brushing his or her teeth (AM) and a second set of samples before dinner (P). The cells were counted using a hemocytometer and Trypan Blue. The numbers of cells, as shown in FIG. 4, are the average of cells for the AM and PM collected samples. As shown in the graph in FIG. 4, the buccal/mammalian cells maintained viability when stored and transported at room temperature for 21 days in the device of the present disclosure.

Stabilizing Microbial DNA:

Purified microbial DNA was normalized to 50 ng/well and loaded on 1% agarose. The stabilized microbial DNA was transported and stored at room temperature for 14 days. The results showing that the DNA was stabilized are depicted in FIG. 5.

Stabilizing Microbial RNA:

Purified microbial RNA was normalized to 20 ng/well and analyzed by gel electrophoresis. The stabilized microbial RNA was transported and stored at room temperature for 14 days. The results showing stabilized RNA are depicted in FIG. 6.

Whole Blood Stabilization:

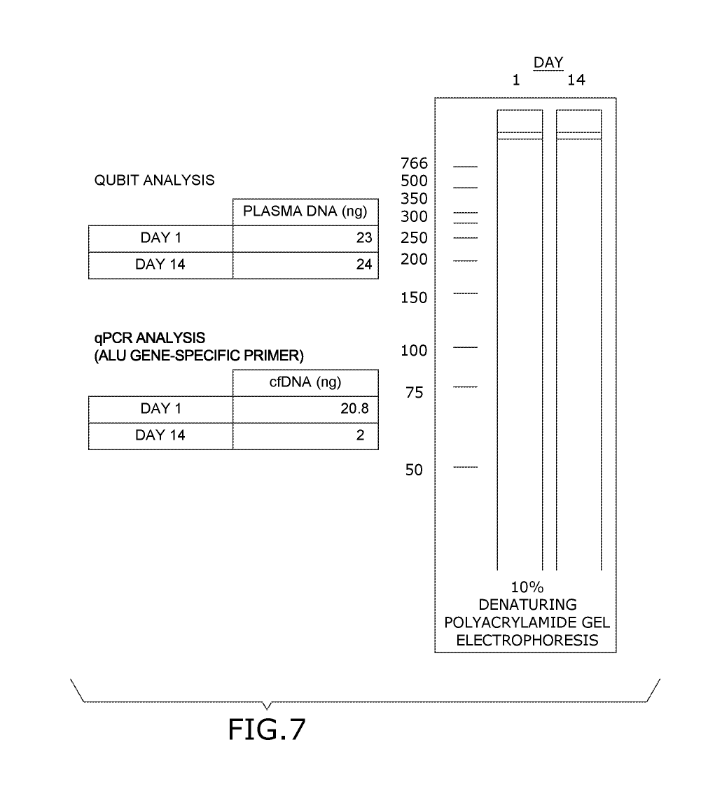

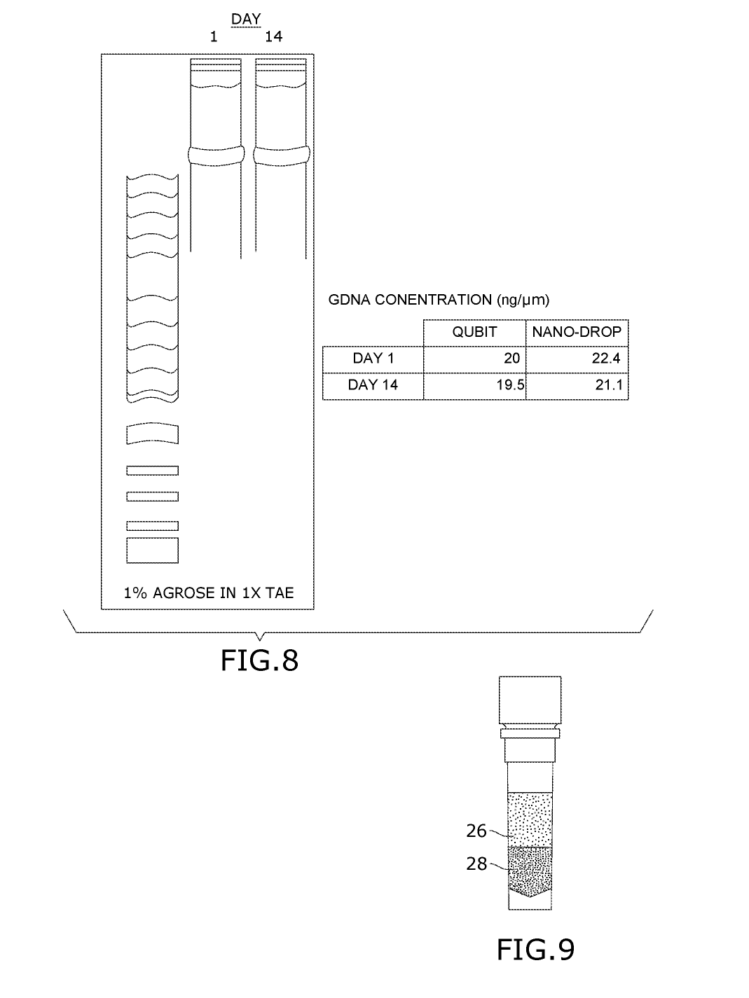

An 8 mL blood draw was transferred to a whole blood preserve tube (i.e., the device of the present disclosure) and was then transported and incubated for 14 days at room temperature before being processed in a certified lab. Plasma was separated and then DNA was extracted from 1 mL of the collected plasma from healthy control using a QIAamp circulating NA kit (150 μL elution). Testing was done at two separate time points—Day 1 and Day 14. The extracted plasma DNA was analyzed using 10% denaturing PAGE, Qubit and qPCR with Alu gene-specific primer sets. QiaAmp mini blood was used to extract gDNA from 200 μL from buffy coat (blood cells derived from plasma) and analyzed on 1% agarose in 1×TAE buffer. The results are shown in FIGS. 7 and 8.

Selective Purification of Microbial Nucleic Acids:

FIG. 9 shows a schematic view of the DNA collection device 12 hours post-collection, the device comprising supernatant fluid 26 (bacterial cells) and the pellet 28 (buccal cells). In this example, 4 swabs were collected from different sides of the mouth. The content of the swabs was released into the vessel, and the device was left sitting on the bench overnight (12 hours) at room temperature. The supernatant 26 is what may be used for recovering bacterial DNA with free or minimal host gDNA contamination.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A device for maintaining the viability of mammalian and microbial cells during ambient temperature transport and storage, the device comprising:
a volume of a buffered sugar alcohol solution comprising a sugar alcohol dissolved in nuclease free water; and
a sealable vessel sized to accommodate the volume of the buffered sugar alcohol solution and a volume of cells to be transported and stored.

2. The device of claim 1, wherein the buffered sugar alcohol solution has a pH of from about 6.0 to about 8.0.

3. The device of claim 1, wherein the sugar alcohol comprises a member selected from the group consisting of polyhydric alcohols, polyalcohols, alditols, and glycitol.

4. The device of claim 3, wherein the sugar alcohol comprises a member selected from the group consisting of glycerol, erythritol, threitol, arabitol, xylitol, ribitol, mannitol, sorbitol, galactitol, fucitol, iditol, inositol, volemitol, isomalt, malitol, lactitol, maltotriitol, maltotetraitol, and polyglycitol.

5. The device of claim 1, wherein the vessel comprises:
a tube; and
a spill-proof cap removably attached to the tube.

6. The device of claim 6, wherein the tube includes a fill line on a surface thereof.
